(19) United States
(12) Patent Application Publication
(10) Pub. No.: US 2025/0221958 A1
(43) Pub. Date: Jul. 10, 2025

(54) Latorias Secret

(71) Applicant: Ronald Clarke, Mobile, AL (US)

(72) Inventor: Ronald Clarke, Mobile, AL (US)

(21) Appl. No.: 18/406,177

(22) Filed: Jan. 7, 2024

(51) Int. Cl. A61K 31/375; A61K 33/00; A61K 33/04; A61K 33/14; A61K 33/16; A61K 33/22; A61K 33/241; A61K 33/30; A61K 33/38

(52) U.S. Cl. CPC A61K 31/375; A61K 33/00; A61K 33/04; A61K 33/14; A61K 33/16; A61K 33/22; A61K 33/241; A61K 33/30; A61K 33/38

(57) ABSTRACT

Bacterial Vaginosis (BV) is caused by the bacterium Gardnerella Vaginalis. It affects 30% of women in the USA ages 14-49. It recurs frequently. Latoria's secret is a natural product, made of elements and compounds forming a solution, a vaginal suppository, and a soap. In Solution, water is 97% of the product. The element and other compounds are 2.5%. The blue color and ascorbic acid are less than 0.5% of the product. The water and other components are mixed to form Latoria's Secret Solution. They are mixed and placed in a vaginal squirt bottle. Latoria's Vaginal Suppository and Soap are made of the compound and a glycerinated gelatin base.

### CROSS-REFERENCE TO RELATED APPLICATIONS

Ser. No. 18/406,177

### STATEMENT REGARDING FEDERALLY SPONSORED RESEARCH OR DEVELOPMENT

### THE NAMES OF THE PARTIES TO A JOINT RESEARCH AGREEMENT

Click here to enter text.

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Click here to enter text.

### STATEMENT REGARDING PRIOR DISCLOSURES BY THE INVENTOR OR A JOINT INVENTOR

### BACKGROUND OF THE INVENTION

### (1) Field of the Invention

Medicine

### (2) Description of Related Art Including Information Disclosed Under 37 CFR 1.97 and 1.98

### BRIEF SUMMARY OF THE INVENTION

### Medication To Treat Bacterial Vaginosis (BV)

Latorias Secret
Purpose: To treat Bacterial Vaginosis (BV)

### INGREDIENTS LIST

Propylene
NaF
Cacl
Mgcl
Naso
Nahco
Srcl
Kbr
Kbr
AgNo
Sulfate
Mgcl
Cacl
Nacl
Ba(No
F
MN(No
HBO
H2O
Cu(No
ZN(No
Pb(No
Blue Color
Ascorbic acid

1. A method of making Latoria's Secret Solution wash, consisting of ingredients mixed as follows, water 97%, Ingredients 2.5%, blue color and ascorbic acid less than 0.5%.

2. A method of making Latoria's Vaginal Suppository, consisting of ingredients plus glycinerated gelatin vaginal base.

3. A method of making Latoria's Secret Soap, consisting of ingredients plus glycinerated gelatin soap base.
